(19) United States
(12) Patent Application Publication
(10) Pub. No.: US 2020/0362408 A1
(43) Pub. Date: Nov. 19, 2020

(54) Multiplexed Method for Detecting DNA Mutations and Copy Number Variations

(71) Applicant: Yan Wang, San Diego, CA (US)

(72) Inventor: Yan Wang, San Diego, CA (US)

(21) Appl. No.: 16/896,231

(22) Filed: Jun. 9, 2020

Related U.S. Application Data
(63) Continuation of application No. PCT/US2018/064715, filed on Dec. 10, 2018.
(60) Provisional application No. 62/596,865, filed on Dec. 10, 2017.

(51) Int. Cl. C12Q 1/6874

(52) U.S. Cl. CPC C12Q 1/6874

(57) ABSTRACT

Disclosed is a method for simultaneously detecting a large number of mutations of different target genes with high specificity and sensitivity. It exploits single-molecule clonal amplification techniques, a hybridization-based decoding technique and a primer extension-based detection method to enable simultaneous measurement of hundreds and thousands of mutation DNAs in a sample. Also disclosed is a method for detecting copy number variation with high sensitivity and accuracy. The invention provides a method for efficiently and accurately counting thousands and millions of sequences from a plurality of target regions, enabling detection of copy number variation at the whole genome, the whole chromosome, sub-chromosomes or single gene level.

### CROSS-REFERENCES AND RELATED APPLICATIONS

This application is a continuation of international application PCT/US2018/064715, filed Dec. 10, 2018, which claims the benefit of priority to U.S. provisional application No. 62/596,865, filed Dec. 10, 2017, the content of which is hereby incorporated by reference in its entirety.

### FIELD OF THE INVENTION

This invention belongs to the field of biotechnologies. In particular, it relates to methods for detecting DNA mutations in a multiplexed format and for detecting copy number variations of a whole chromosome or subsections of it.

### BACKGROUND OF THE INVENTION

Many mutant variants of nucleic acids such as Single Nucleotide Polymorphisms (SNPs), insertions/deletions, gene fusions and copy number variants are implicated in a variety of medical situations, including genetic disorders, susceptibility to diseases, predisposition to drug resistance, and progression of diseases. Methods and technologies for effectively detecting mutant variants thus play an increasingly important role in clinical applications. In many clinical settings, it is required to detect and quantitate disease-associated rare mutant variants against a high background of wild-type sequences or alternative variants. For example, circulating cell-free DNA (cfDNA) in bloodstream, so called “liquid biopsy”, is an invaluable source for non-invasively detecting somatic mutations associated with cancer prognosis and therapeutic efficacy. Some tumor-related mutants in cfDNA samples are found to have an allele frequency as low as 0.01%, which presents a great challenge for developing technologies to detect such low frequency mutant alleles. Another important application of liquid biopsy is detection of the small fraction of fetal cfDNAs under the background of maternal DNAs, which is essential for detecting prenatal genetic disorders. In addition, the starting materials in clinical samples are very limited (e.g. 5-20 ng total DNA) and multiple diagnostic tests are needed. These present a great challenge for developing technologies for detection of low frequency alleles with high sensitivity and specificity as well as methods that can be applied in a highly multiplexed format.

The most straight-forward method for detecting a mutation is direct hybridization with mutation specific probes (e.g. microarray assays). Microarray assays use hybridization of allele specific probes to differentiate mutant alleles from wild-type alleles and can simultaneously measure hundreds and thousands of different mutations. Although these methods have been used in detecting germline nucleotide mutations and copy number variations, the methods often suffer from low specificity and low sensitivity of the probes, and can have high background and high false detection rate. They usually do not possess sufficient specificity and sensitivity to satisfy the stringent requirements of detection of somatic mutations and fetal genetic abnormality in cfDNA samples.

PCR-based detection techniques have higher specificity and sensitivity than that of microarrays, but these methods are difficult to be applied in highly multiplexed formats. A commonly used PCR-based method uses duel labeled mutant specific Taqman® probes. Taqman® probe is an oligonucleotide consisting of a fluorophore at the 5′ end and a quencher at the 3′ end. When the fluorophore and the quencher are in close proximity, no fluorescent signal is emitted. During the extension stage of a PCR, Taqman® probes anneal to the mutant sequence and a fluorescent signal is released when the 5′ end of Taqman® probes is cleaved by a Taq polymerase enzyme, thus detecting the mutant sequence. The Taqman® assay generally has higher specificity and sensitivity than that of direct hybridization, and can be applied to detect nucleotide mutations and copy number variation of a target region. However, the design and optimization of specific Taqman® probes for each mutation detection is still a challenging and time-consuming task. The cost of Taqman® probes are quite high due its complex structure. It is also very difficult to develop multiplexed Taqman® assays due to limited availability of different types of fluorophores.

Allele-specific polymerase chain reaction (AS-PCR) is another widely used PCR-based method for selectively amplifying and detecting mutant variants (Wu D Y, Ugozzoli L, Pal B K, Wallace R B, Proc Natl Acad Sci USA 1989; 86:2757-2760; Chen X, and Sullivan P F, The Pharmacogeonomics Journal 2003; 3:77-96). AS-PCR uses allele-specific PCR primers complementary to the target polymorphic site of the mutant allele to selectively amplify the mutant variant. The selectivity and specificity of AS-PCR is largely dependent on the selectivity of DNA polymerase that extends primers at a much lower efficiency with a mismatched 3′ end than that with a matched 3′ end. However, exponential PCR amplification makes quick decay of this discriminating power and significant mismatched amplification often occurs. The discrimination power of this method is also affected by the ratio of wild-type vs. mutant allele and the sequences around the polymorphic base. It is very difficult to perform AS-PCR in a small-scale multiplexed way, let alone to perform hundreds and thousands of AS-PCR at the same time.

Detection of fetal chromosome abnormality in cell-free circulating DNA from maternal blood has important clinical applications. For example, birth defects such as Down Syndrome, Edward's Syndrome and Patau Syndrome are caused by additional copy of chromosome 21, 18 and 13, respectively. However, detection of the small amount of fetal DNA (usually <4% of total circulating DNA) under the background of maternal DNA poses a stringent requirement on the specificity and the sensitivity of the detection technology. The microarray-based detection methods lack the sensitivity, accuracy and specificity to resolve small differences required in prenatal DNA tests. The current technologies use high-throughput DNA sequencing technologies to detect fetal genetic disorders in maternal cfDNA samples. With high sequencing coverage, the high-throughput DNA sequencing technology has the resolution power to detect fetal chromosome abnormalities. But it requires multiple runs of sequencing and complex and sophisticated data analysis. The turnaround time takes up to several days. It is too expensive and time consuming to be routinely used in clinical tests.

These existing detection technologies are not ideal. To satisfy the requirement of high sensitivity and specificity in clinical tests, especially tests of plasma cfDNA samples where sample materials are limited, there is a need for developing reliable and robust technologies that allow specific and sensitive detection of rare mutants with low allele frequencies, and that can be applied for multiplexed detection of a large number of target sequences at manageable costs. There is a great need for developing technologies for detecting copy number variation with high sensitivity and accuracy that can be applied to prenatal DNA tests in maternal cell-free circulating DNA samples. The present invention satisfies this need and provides other benefits as well.

### SUMMARY OF THE INVENTION

The present invention provides a method for simultaneously detecting a large number of DNA mutations of different target sequences with high sensitivity and specificity. Thousands and millions of DNA molecules in a sample are first captured to a solid surface and are locally amplified to form immobilized DNA clusters of identical sequences. The DNA clusters having the target sequences are then identified by a decoding algorithm using sequential hybridization with a set of decoder sequence pools. The mutant sequences can be detected during the decoding process or by a mutant specific extension performed after the decoding process.

The invention uses mutant primer specific DNA extension to detect and enumerate DNA mutation molecules directly captured from a DNA sample, which offers a detection method with high specificity, high sensitivity and high accuracy. Combined with a decoding technique, it can simultaneously measure hundreds and thousands of immobilized mutation sequences without making hundreds and thousands of labeled probes, which greatly reduces material costs and the detection variation caused by varied hybridization efficiency of different labeled probes. The invented method can also be applied to detect copy number variation of a whole chromosome and subsections of it by efficiently counting a large number of sequences from different chromosomes or different subsections of a chromosome.

In one embodiment, the present invention provides a method for simultaneously enumerating a plurality of target sequences in a DNA sample, comprising the steps of: a) performing a single-molecule clonal amplification on the DNA sample to obtain a large number of immobilized DNA clusters, each having an identical DNA sequence and being spatially separated from one another with a random distinguishable address; b) decoding the identity of the DNA clusters having target sequences by use of a hybridization decoding process with a set of decoder sequence pools; and c) enumerating DNA clusters having target sequences, thereby obtaining the number of each target sequence in the DNA sample. The hybridization decoding process comprises the steps of: a) providing a decoder sequence specific for each target sequence, wherein each decoder sequence has N different labeling states, wherein N is at least 2; b) designing a M-bit identification code to uniquely represent each decoder sequence, wherein M rounds of decoding hybridizations are to be performed to decode T types of different target sequences, and the value of i^{th} bit ( i=1, 2, . . . M) of the M-bit identification code of a decoder sequence defines the labeling state of the decoder sequence used in the decoder sequence pool for the i^{th} round decoding hybridization, wherein T is the total number of different types of target sequences and M is no less than ┌log_{N}T┐; c) making a set of M pools of decoder sequences according to the M-bit identification codes; d) performing M rounds of sequential decoding hybridizations with the decoder sequence pool set and the DNA clusters in an order defined by the M-bit identification codes; and e) recording the labeling state of each DNA cluster in each round of decoding hybridization to decode the identity of DNA clusters based on the M-bit identification code for each decoder sequence.

In some embodiment, different alleles of a target sequence are recognized by one target sequence specific decoder sequence. In some embodiment, different alleles of a target sequence are recognized by different allele specific decoder sequences.

In some embodiment, the decoder sequence is linked to a detectable label. The detectable label is selected from a fluorescent, a chemiluminescent or a biotin label. The labeling state of a decoder sequence is represented by the type of the detectable label linked to the decoder sequence. Additionally, the labeling state of a decoder sequence can be represented by no presence of the decoder sequence.

In some embodiment, the decoder sequence comprises two oligonucleotides complementary to adjacent sections of its target sequence, wherein the two oligonucleotides are respectively end labeled with a donor and a acceptor fluorophore that form a FRET pair.

In some embodiment, the decoder sequence has two labeling states, represented by the presence and the absence of the decoder sequence, respectively.

In some embodiment, each decoder sequence pool comprises a selected combination of decoder sequences, wherein the presence of a decoder sequence is designated as 1 and the absence of a decoder sequence is designated as 0 in the M-bit identification code, and each decoder sequence is represented by a M-bit binary identification code.

In some embodiment, the presence of a decoder sequence is detected by a label directly linked to the decoder sequence. The label linked to the decoder sequence is a biotin, a fluorophore, or a chemiluminescent moiety. The decoder sequences can be labeled with the same or different fluorophores.

In some embodiment, the decoder sequence comprises of two oligonucleotides complementary to adjacent sections of its target sequence, wherein the two oligonucleotides are respectively end labeled with a donor and an acceptor fluorophore that form a FRET pair.

In some embodiment, the decoder sequences are unlabeled, and the presence of a decoder sequence is detected by decoder sequence mediated DNA polymerization.

In some embodiment, the presence of a decoder sequence is detected by using decoder sequence mediated DNA polymerization to make an labeled extension strand. A labeled dNTP is added during decoder sequence mediated DNA polymerization to make a labeled extension strand, wherein the labeled dNTP comprises a fluorescent, a chemiluminescent or a biotin moiety.

In some embodiment, the DNA cluster annealed to a decoder sequence is labeled by detecting a physical or chemical change generated by the decoder sequence mediated DNA polymerization. The physical or chemical change is selected from pyrophosphate, hydrogen ion and temperature change generated during detection sequence mediated DNA polymerization.

In some embodiment, it further comprises the steps of: a) denaturing and removing the decoder sequences from the DNA clusters; b) annealing a plurality of detection sequences to respective target sequences within the DNA clusters in a detection hybridization; c) labeling DNA clusters annealed to detection sequences; and d) enumerating labeled DNA clusters having target sequences. The decoder sequence and the detection sequence of a target sequence can be the same or different.

In some embodiment, the decoder sequence is target sequence specific and the detection sequence is allele specific. The target specific decoder sequence can recognize common sequences shared by different alleles of the target sequence. The allele specific detection sequence recognizes allele specific sequence of the target sequence, for example, a wild-type allele or a mutant allele.

In some embodiment, the method to label DNA clusters in the decoding hybridization and the detection hybridization is different. For example, a decoder sequence with a fluorescent label is used to label DNA clusters in the decoding hybridization. Unlabeled detection sequence uses detection sequence mediated DNA polymerization to label DNA clusters in the detection hybridization.

In some embodiment, the method is used for detection of copy number variation of the target sequences. The target sequences are divided into a first and second part, wherein the first part contains sequences to be tested for the presence of copy number variation, and the second part contains reference sequences that are known to have no copy number variation, and wherein the presence of a copy number variation for a target sequence is detected when the number of the target sequence is significantly different from those of reference sequences.

In some embodiment, the method is used for detecting copy number variation of a plurality of different target regions of a DNA sample. The decoder sequences are divided into a plurality of first decoder sequences, each complementary to a different target sequence within one of the target regions, and providing a plurality of second decoder sequences, each complementary to a different target sequence within one of reference regions that are known to have no copy number variation, wherein the first and the second decoder sequences are combined to use for decoding the DNA Clusters, and wherein the numbers of target sequences of a target region and the numbers of target sequences of reference regions are compared to determine if the target region has a copy number variation.

In some embodiment, the average number of all the target sequences of a target region and the average number of all the target sequences of a reference region is used to determine if the target region has a copy number variation.

In some embodiment, target sequences of a target region are grouped into a sequence bin of certain length, and the average number of target sequences in each sequence bin of the target region and the average number of target sequences in each sequence bin of the reference region are used for determination of the presence of copy number variation in the target region. The length of a sequence bin can be at least 10 kb, 100 kb, 1 Mb, or 10 Mb.

In one embodiment, the present invention provides a method for simultaneously enumerating an allelic form of a plurality of different target sequences in a DNA sample, comprising the steps of: a) performing a single-molecule clonal amplification on the DNA sample to obtain a large number of immobilized DNA clusters of identical DNA sequences, wherein each DNA cluster is spatially separated from one another and has a random distinguishable address; b) decoding the identity of the DNA clusters having target sequences by use of sequential hybridization with a set of target sequence specific decoder sequence pools; c) annealing a plurality of detection primers, which are specific to an allelic form of the target sequences, to respective complementary sequences within the DNA clusters; d) labeling the DNA clusters annealed to detection primers by using the detection primer mediated DNA polymerization to make extension strands; and e) enumerating labeled DNA clusters with the decoded identity, thereby simultaneously counting the number of DNA molecules of the allelic form for each target sequence.

In some embodiment, the detection primer is specific to a mutant allele of the target sequence, which is different from the decoder sequence that recognizes the common sequence shared by the wild-type and the mutant alleles of the target sequence. This method can be used to enumerate mutant alleles of different target sequences in the DNA sample.

In some embodiment, the method further comprising the steps of: a) denaturing and removing the extension strands from the DNA clusters, and annealing a plurality of wild-type detection primers of the target sequences to respective complementary sequences within the DNA clusters; b) labeling the DNA clusters annealed to wild-type detection primers using the wild-type detection primer mediated DNA polymerization; c) enumerating labeled DNA clusters with the decoded identity, thereby simultaneously counting the number of DNA molecules of the wild-type allele for each target sequence; and d) calculating a mutant allele frequency for each target sequence by dividing the number of the mutant allele with the total number of the mutant and the wild-type allele.

In some embodiment, the detection primer is specific to a wild-type allele of the target sequence and the method can be used to enumerate wild-type alleles of different target sequences in the DNA sample.

In some embodiment, the decoder sequence for a target sequence is the same as the detection primer for the same target sequence. In another embodiment, the decoder sequence for a target sequence is different from the detection primer for the same target sequence. Using different decoder and detection sequences of target sequences can further verify the accuracy of the decoding process and increase the detection specificity.

In some embodiment, the method is used for detection of the presence of copy number variation of a target sequence as compared to a reference sequence. The target and the reference sequence can be divided into a plurality of subsequences, respectively. The subsequences of the target and the reference sequence can be decoded and enumerated using the methods described herein. The average number of the subsequences can be used as a representation of the copy number of the respective parent sequence. The presence of copy number variation for a target sequence is detected when the copy number of the target sequence is significantly different from that of the reference sequence.

In some embodiment, the DNA clusters annealed to a detection primer are labeled by using detection primer mediated DNA polymerization to make a labeled extension strand. In some embodiment, a labeled dNTP is added during detection primer mediated DNA polymerization to make the labeled extension strand. The labeled dNTP can comprise, for example, a fluorescent, a chemiluminescent or a biotin label.

In some embodiment, the DNA clusters annealed to a detection primer are labeled by detecting a physical or chemical change generated by detection primer mediated DNA polymerization. The physical or chemical change can be selected from pyrophosphate, hydrogen ion and temperature change generated during detection primer mediated DNA polymerization.

In some embodiment, the decoding process uses a set of selected decoder sequence pools in sequential hybridizations to identify each DNA cluster containing a target sequence, comprising the steps of: a) providing a decoder sequence specific for each target sequence, wherein each decoder sequence has N different labeling states, wherein N is at least 2; b) designing a M-bit identification code to uniquely represent each decoder sequence, wherein M rounds of decoding hybridizations are to be performed to decode T types of different target sequences, and the value of i^{th} bit (i=1, 2, . . . M) of the M-bit identification code of a decoder sequence defines the labeling state of the decoder sequence used in the decoder sequence pool for the i^{th} round decoding hybridization, wherein M is ┌log_{N}T┐, and T is the total number of different types of target sequences; c) making a set of M decoder sequence pools according to rules embedded in the M-bit identification code for each decoder sequence; d) performing M rounds of sequential decoding hybridizations with the decoder sequences pool set and the DNA clusters in an order defined by the M-bit identification codes; and e) recording the labeling state of each DNA cluster in each round of decoding hybridization to decode the identity of DNA clusters based on the M-bit identification code for each decoder sequence. In some embodiment, an additional (M+1)^{th} round hybridization with a selected decoder sequence pool can be used to verify the decoding accuracy.

In some embodiment, the labeling state of a decoder sequence is represented by the type of the fluorophore linked to the decoder sequence, wherein the number of labeling states can be selected from 2, 3, 4, 5, 6, 7 or more.

In some embodiment, the labeling state of a decoder sequence is represented by the type of the fluorophore linked to the decoder sequence, and the non-fluorescence can also be used as one labeling state. For example, a red fluorophore, a green fluorophore, and the non-fluorescence can be counted as a total of three labeling states.

In some embodiment, the annealing of a decoder sequence to its complementary target sequence is detected by fluorescence resonance energy transfer (FRET). For example, the decoder sequence comprises two oligonucleotides complementary to adjacent sections of its target sequence, wherein the two oligonucleotides are respectively end labeled with a donor and an acceptor fluorophore that form a FRET pair.

In some embodiment, a decoder sequence has two different labeling states, represented by the presence (e.g. the fluorescent state) and the absence (e.g. the non-fluorescent state) of the decoder sequence, respectively. To use a digital form of representation, each decoder sequence is uniquely represented by a M-bit binary identification code, wherein the presence of a decoder sequence is designated as 1 and the absence of a decoder sequence is designated as 0 in the M-bit identification code. Each decoder sequence pool comprises a selected combination of decoder sequences that is determined by the M-bit identification codes for all the decoder sequences.

In some embodiment, the presence of a decoder sequence is detected by a label directly linked to the decoder sequence. The label linked to the decoder sequence is a biotin, a fluorophore, or a chemiluminescent moiety. In some embodiment, all the decoder sequences are labeled with the same fluorophore. In some embodiment, the decoder sequences are labeled with different fluorophores. In some embodiment, the decoder sequence comprises of two oligonucleotides complementary to adjacent sections of its target sequence, wherein the two oligonucleotides are respectively end labeled with a donor and an acceptor fluorophore that form a FRET pair.

In some embodiment, decoder sequences are unlabeled, and the presence of a decoder sequence is detected by decoder sequence specific DNA extension. In some embodiment, an unlabeled decoder sequence pool, a DNA polymerase, and a dNTP mix with a fluorescent nucleotide are added during a decoding hybridization, and the presence of a decoder sequence in a DNA cluster is detected by the decoder sequence specific extension that makes a labeled extension strand. In some embodiment, one, two, three or four types of nucleotides in the dNTP mix are substituted by respective fluorescent nucleotides.

In some embodiment, nucleotides with two different fluorophores are used to label decoder sequence specific extension strands in alternate rounds of decoding hybridizations. In this paradigm, one hybridization is labeled with one fluorophore and the subsequent hybridization is labeled with another fluorophore with a different color. By this way, residual labeling from previous hybridization can be easily detected, thus reducing error rate.

In some embodiment, an unlabeled decoder sequence pool, a DNA polymerase, and a dNTP mix of four natural nucleotides are added during the decoding hybridization. The presence of a decoder sequence in a DNA cluster is detected by recording a chemical or physical change generated by the decoder sequence specific DNA extension. The chemical or physical change generated by the decoder sequence specific DNA extension is selected from pyrophosphates, H⁺ ions, and temperature change.

In some embodiment, the present invention provides a method for simultaneously enumerating a plurality of different target sequences in a DNA sample, comprising the steps of: a) performing a single-molecule clonal amplification on the DNA sample to obtain a large number of immobilized DNA clusters of identical DNA sequences, wherein each DNA cluster is spatially separated from one another and has a random distinguishable address; b) providing a plurality of decoder sequences, each specific for a target sequence, wherein each decoder sequence has two labeling states, the presence of the decoder sequence and the absence of the decoder sequence, which are assigned digital values of 1 and 0, respectively; c) designing a M-bit binary identification code to uniquely represent a decoder sequence, wherein M rounds of decoding hybridizations are to be performed to decode T types of decoder sequences, and the value of i^{th} bit (i=1, 2, . . . M) of the M-bit identification code defines the labeling state of the respective decoder sequence used in the decoder sequence pool for the i^{th} round decoding hybridization, wherein M is ┌log₂T ┐, and T is the number of different types of decoder sequences; d) making a set of M decoder sequence pools according to the M-bit binary identification codes; e) performing M rounds of sequential decoding hybridizations with the decoder sequences pool set and the DNA clusters in an order defined by the M-bit identification codes, wherein labeling states of DNA clusters in each round of decoding hybridization are determined by decoder sequence mediated DNA polymerization to make extension strands; and f) recording the labeling state of each DNA cluster in each round of decoding hybridization to decode the identity of DNA clusters and count the number of each target sequence in the DNA sample.

In some embodiment, the target sequence is a mutant sequence of a target gene and the decoder sequence comprises a mutant specific sequence. This method can be used to directly detect mutant sequences of different target sequences.

In some embodiment, the target sequences are separated into a first part of target sequences comprising mutant sequences of target genes and a second part of target sequences comprising corresponding wild-type sequences of the target genes. Accordingly, the decoder sequences are separated into the first part of decoder sequences comprising mutant specific sequences and the second part of decoder sequences comprising wild-type specific sequences.

In some embodiment, the presence of a decoder sequence is determined by decoder sequence mediated DNA polymerization to make a labeled extension strand. In some embodiment, a labeled dNTP is added during decoder sequence mediated DNA polymerization to make a labeled extension strand. The labeled extension strand comprises a fluorescent, a chemiluminescent or a biotin label. In some embodiment, one, two, three or four types of fluorescent nucleotides are added during decoder sequence mediated DNA polymerization.

In some embodiment, the presence of a decoder sequence is determined by detecting a physical or chemical change generated by decoder sequence mediated DNA polymerization. The physical or chemical change is selected from pyrophosphate, hydrogen ion and temperature change generated during decoder sequence mediated DNA polymerization.

In some embodiment, the method is used for detection of copy number variations. The target sequences are separated into a first part containing sequences to be tested for presence of copy number variations and a second part containing reference sequences known to have no copy number variation. The presence of a copy number variation for a particular target sequence is detected when the number of the target sequence in the DNA sample is significantly different from those of reference sequences.

In some embodiment, the present invention provides a method for detecting copy number variation of a plurality of target regions in a DNA sample, comprising the steps of: a) providing a plurality of first decoder sequences, each complementary to a different target sequence within one of the target regions, and providing a plurality of second decoder sequences, each complementary to a different target sequence within one of reference regions; b) performing a single-molecule clonal amplification on the DNA sample to obtain a large number of immobilized DNA clusters of identical DNA sequences, wherein each DNA cluster is spatially separated from one another and has a random distinguishable address; c) combining the first and the second decoder sequences to decode DNA clusters having sequences complementary to the first or second decoder sequences using the decoding method described above; d) counting the number of each target sequence of target regions and the number of each target sequence of reference regions; and e) comparing the numbers of target sequences of target regions and the numbers of target sequences of reference regions to determine if a target region has a copy number variation. The presence of copy number variation of a target region is detected when the number of target sequences of target regions is significantly different from those of the reference regions.

In some embodiment, the number of the first decoder sequences or the second decoder sequences is at least 20, 30, 50, 100, 200, 500, 1000, 10000, 100000, 1000000, or 10000000.

In some embodiment, the target region is a single gene, a cDNA sequence, a genomic region of interest, a chromosome or a whole genome.

In some embodiment, the target region is a chromosome and the target sequences are selected to be evenly distributed along the chromosome. In some embodiment, the target sequences are selected from stable regions of a chromosome.

In some embodiment, the average number of all the target sequences of a target region and the average number of all the target sequences of a reference region is used to determine if the target region has a copy number variation. A copy number variation of a target region is detected when the average number of all the target sequences of the target region is significantly different from that of a reference region.

In some embodiment, target sequences of a target region are grouped into a sequence bin of certain length, and the average number of target sequences in each sequence bin of the target region and the average number of target sequences in each sequence bin of the reference region are used for determination of the presence of a copy number variation in the target region. The length of a sequence bin is at least 10 kb, 100 kb, 1 Mb, or 10 Mb.

### BRIEF DESCRIPTION OF DRAWINGS

FIG. 1. A schematic diagram of the invention with a) the clonal amplification, b) the decoding and c) the mutant detection steps. a) Tagged DNA molecules are randomly captured to a solid support and clonally amplified to make DNA clusters, each having identical sequences. b) DNA clusters are decoded to identify the DNA clusters having target sequences (labeled as No. 1, 2, and 3 target sequences). c) The mutant specific extension is used to detect the presence of mutations within the target sequence DNA clusters (each mutant sequence is labeled with a star).

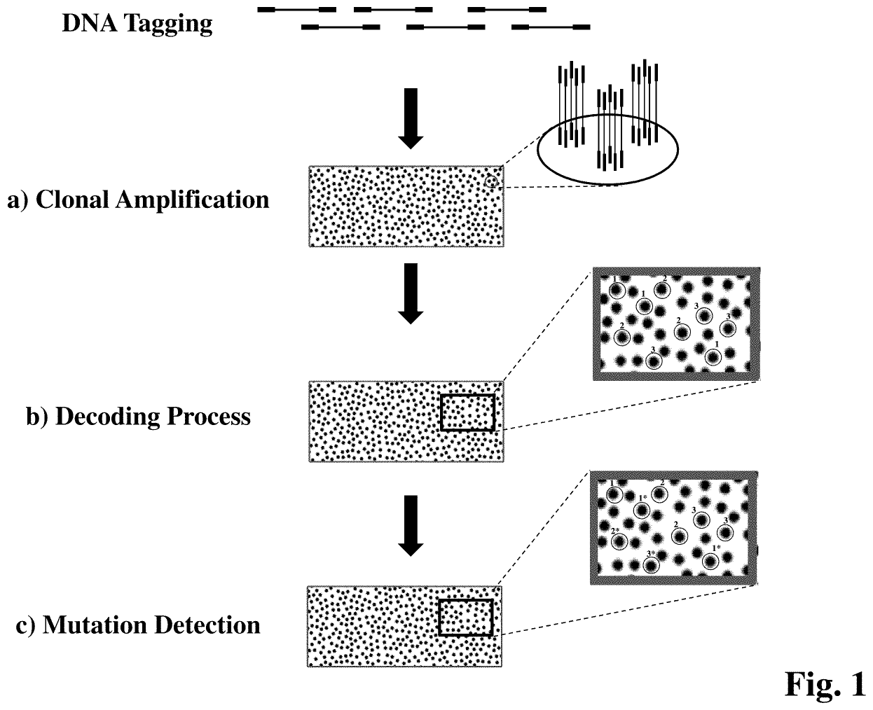

FIG. 2. Examples of using the invented method for detection of multiple mutations and copy number variations. In one paradigm, the decoding process and the detection process are separated in two stages to detect mutations or copy number variations. In another paradigm, the decoding process and detection process are combined in one stage.

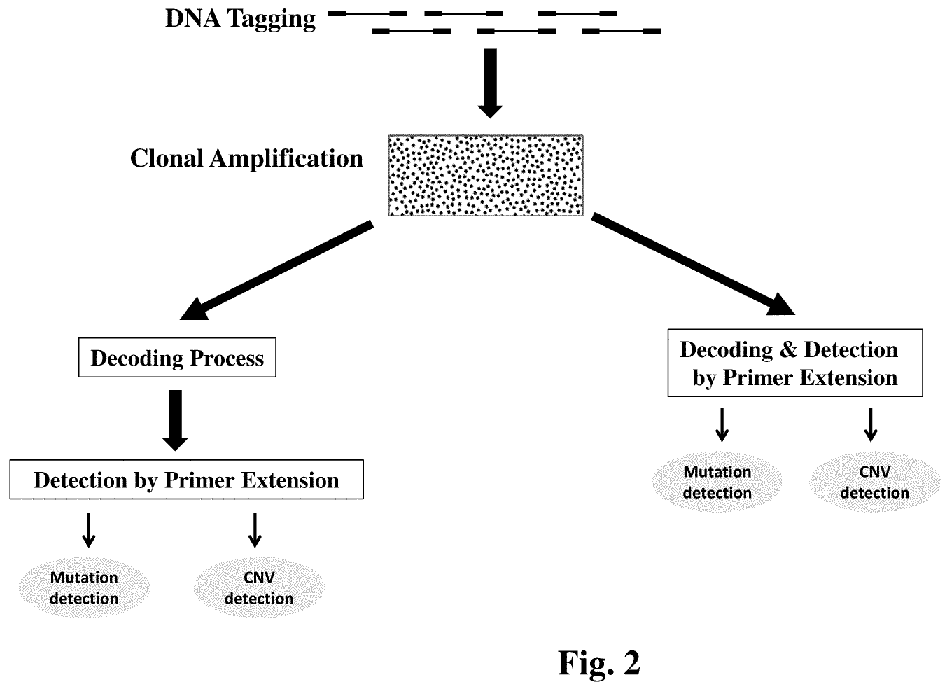

### DETAILED DESCRIPTION

Definitions: Unless otherwise defined, all technical and scientific terms used herein have the same meaning as commonly understood by one of the ordinary skills in the art to which this invention belongs.

The term “a” and “an” and “the” as used to describe the invention, should be construed to cover both the singular and the plural, unless explicitly indicated otherwise, or clearly contradicted by context Similarly, plural terms as used to describe the invention, for example, nucleic acids, nucleotides and DNAs, should also be construed to cover both the plural and the singular, unless indicated otherwise, or clearly contradicted by context.

The term “DNA sample” as used herein, refers to a population of DNA sequences obtained from any sources. For example, a nucleic acid sample may be prepared from cells, tissues, organs, soils, air, water, fossils and any other biological and environmental sources. Particularly, a nucleic acid sample may be prepared from a patient's tissue, a body fluid, or a cell sample such as urine, lymph fluid, spinal fluid, synovial fluid, serum, plasma, saliva, skin, stools, sputum, blood cells, tumor cells/tissues, organs, and also samples of in vitro cell culture constituents, which can be used for molecular diagnostic and prognostic purpose. A DNA sample may include, but not limited to, circulating cell-free DNA, genomic sequences, subgenomic sequences, chromosomal sequences, PCR products, amplicon sequences and cDNA sequences. The DNA sequences can be linked to preselected sequence tags on one or both ends. The sequence tags are predesigned sequences that are non-complementary to all the sequences in the nucleic acid sample, which can be used as anchor sequences to anneal the DNA sequences to complementary oligos attached to a solid surface. When the starting material is RNA, e.g. mRNA, rRNA, whole transcriptome, miRNA, and smRNA, the RNA molecules can be converted to DNAs and used in the invention.

The term “target gene/sequence”, as used herein, refers to a region or locus of DNA or RNA that is of particular interest to the user and is the sequence to be detected or measured. A target gene may be a DNA coding region of a protein, a regulatory region of a gene, and a region of an mRNA, an smRNA, a miRNA or an rRNA. The target gene usually has various forms in terms of the nucleic acid sequence. The most common and prevalent form in a population is the “wild-type sequence”, “wild-type allele” or “wild-type gene”. The other forms having mutations relative to the wild-type sequence are considered “mutant variants”, “mutation DNA” or “mutant allele”. The mutations can have different types, including, for example, nucleotide substitutions, insertions, deletions, gene fusions, and any combination thereof. The location where the sequence divergence occurs between a mutant variant and a wild-type sequence is a mutated or polymorphic region. A mutated region, as used herein, refers to a continuous section of a sequence that includes the actual locus of nucleotide substitution, insertion, deletion, and gene fusion. A mutant variant can have more than one mutated region compared to a wild-type sequence.

The term “wild-type gene/sequence”, as used herein, refers to a standard “normal” allele sequence of a target gene of interest, in contrast to a non-standard, “mutant” allele sequence. Generally, the wild-type gene/sequence is the one with the highest gene frequency in nature, and is associated with normal phenotypes. The wild-type gene/sequence used herein particularly refers to the polymorphic region where the divergence between the wild-type sequence and the mutant sequence occurs. The wild-type sequence and mutant sequence/DNA mutation refer to the respective sequences at the same polymorphic site of the target gene.

The term “DNA mutation”, as used herein, refers to a non-standard, “mutant” allele sequence of a target gene, in contrast to a standard “normal” allele sequence (wild-type sequence). In particular, DNA mutation refers to the change of nucleotide sequences in comparison to the corresponding wild-type sequence. A DNA mutation can be a single nucleotide substitution, a multi-nucleotide substitution, an insert of one or more nucleotides, a deletion of one or more nucleotides, a gene fusion between the target gene and another different gene, an altered DNA methylation pattern, or any combination of the above. In some instances, both the change of the nucleotides and the location of the DNA mutation are known; in other instances, only the location of DNA mutation is known, but the actual change of nucleotides is not known. The detection of a DNA mutation refers to detection the presence of such DNA mutation or determination of the number of the mutant molecules in a sample.

The term “single-molecule clonal amplification”, as used herein, refers to an amplification process for generating a large number of DNA sequences from one single DNA molecule to form a localized DNA cluster. This technique uses one single DNA molecule as a template and performs PCR amplification to generate thousands and millions of copies of DNA sequences in a localized region. At least a part of the PCR primers are immobilized to a solid support, which allows the generated DNA molecules to be immobilized to a local cluster so as to form a distinguishable “clone”. In some embodiment, the generated DNA cluster comprises DNA duplexes; in other embodiment, the generated DNA cluster comprises single-stranded DNAs. Examples of the single-molecule clonal amplification technique include Bridge-PCR technique (U.S. patent application Ser. No. 11,725/597) and bead-based emulsion PCR technique (M. Margulies et al. Nature. 2005; 437(7057): 376-380; and M.Y. Xu, et al. Biotechniques. 2010; 48(5): 409-412). For Bridge amplification technique, a single DNA molecule is amplified to form a DNA cluster by in situ PCR using primers attached to a solid surface such as a glass slide. Each DNA cluster is a physically separated “clone” consisting of identical DNA sequences. For emulsion PCR-based clonal amplification, single DNA strands are attached to microbeads which are clonally amplified in emulsion droplets. The clonal amplification of single molecules can also be performed in separate micro-wells.

The term “DNA clusters”, as used herein, refers to a localized cluster of DNA molecules having identical sequences which is generated from a single-molecule clonal amplification. The DNA cluster comprises identical single-stranded or double-stranded DNA sequences that are attached to a solid support. For example, the DNA clusters can be generated on spots of a glass slide or be attached to microbeads, micro-wells or other microparticles.

The term “detection sequence/primer”, as used herein, refers to a DNA sequence that is complementary to a target sequence or one allelic form of a target sequence. It can be designed to recognize a common region shared by all the allelic forms of a target sequence, thus named as target sequence specific detection sequence/primer. It can also be designed to specifically recognize to one allelic form and differentiate it from other allelic forms of the target sequence, thus named as allele specific detection sequence/primer. For example, the detection primer can be designed to specifically recognize a mutant allele or a wild-type allele of a target sequence. The detection primer contains allele specific nucleotide at its 3′ end so that it forms a matched 3′ end pair only with the particular allele. The detection primer specifically binds to the particular allele and functions as a primer to direct DNA polymerization using the particular allele as the DNA template. When the detection primer is designed to specifically recognize a mutant allele, it is interchangeably referred as a “mutant/mutation detection primer” or “mutant/mutation specific primer”. When the detection primer is designed to specifically recognize a wild-type allele, it is referred as a “wild-type detection primer” or “wild-type specific primer”.

The term “mutation specific strand”, as used herein, refers to a DNA sequence generated by polymerase extension of a mutation specific primer against a mutant sequence template in contrast to wild-type sequences. In some embodiment, the mutant specific strand is incorporated with labeled nucleotides that can be directly detected. The detection of the labeled mutant specific strand indicates the presence of the mutant sequence in the particular DNA cluster.

The term “wild-type specific strand”, as used herein, refers to a DNA sequence generated by polymerase extension of a wild-type specific primer against a wild-type sequence template in contrast to mutant sequences. In some embodiment, the wild-type specific strand is incorporated with labeled nucleotides that can be directly detected. The detection of the labeled wild-type specific strand indicates the presence of the wild-type sequence in the particular DNA cluster.

The term “stable region of a chromosome”, as used herein, refers to a genomically and genetically stable region on a chromosome that has no copy number variation in a normal diploid genome, and have few SNP, insertion, deletion, gene fusion or other genetic mutations. The copy number of the stable region should represent the copy number of the chromosome it belongs to. For example, in high throughput sequencing data, the sequence reads of stable regions on different chromosomes should be statistically the same in normal diploid subjects. If copy numbers of stable regions on a target chromosome are consistently and statistically higher or lower than those of reference chromosomes, the target chromosome is expected to have a chromosome abnormality

The term “decoding process”, as used herein, refers to a process to identify all the DNA clusters having target sequences, including identification of the location of such DNA cluster and the particular target sequence that it contains. The target sequences are uniquely identified by a target sequence specific decoder sequence that is complementary to part of the target sequence and can specifically anneal to the target sequence in the hybridization process. A target sequence is identified as a sequence containing a complementary sequence of the corresponding decoder sequence or detection sequence. The DNA clusters having target sequences may only be a part of all the DNA clusters immobilized on a solid support.

The term “decoder sequence”, as used herein, refers to a polynucleotide sequence that is designed to be complementary to part of a target sequence and is used to identify a target sequence. For example, a first decoder sequence identifies a first target sequence that comprises a sequence complementary to the first decoder sequence. A second decoder sequence identifies a second target sequence that comprises a sequence complementary to the second target sequence. A selected combination of different decoder sequences are used in the decoding hybridizations to locate DNA clusters having target sequences. In some embodiment, a decoder sequence is different from a mutation detection sequence or wild-type detection sequence in that the decoder sequence does not contain the nucleotide(s) at the mutation site/locus. A decoder sequence is chosen to be in close proximity to the mutation or wild-type detection sequence. A decoder sequence may be overlapped with a mutation detection sequence. For example, a decoder sequence may overlap with a mutation sequence at the 5′ sequence but lacks the mutation nucleotide(s) at the 3′ end. A decoder sequence identifies target sequences that can be in a form of a mutant or wild-type allele. In some embodiment, the mutant or wild-type specific sequences are used as decoder sequences in the decoding process. In this way, the DNA clusters having a mutant or wild-type sequence can be directly identified after the decoding process.

The term “labeling state”, as used herein, refers to physical or chemical state associated with a decoder sequence or a DNA cluster that can be distinguished by a physical or chemical method. For example, a decoder sequence can be labeled with a green, a red, or a blue fluorophore. The decoder sequence thus has three distinguishable labeling states: green, red or blue fluorescence. In addition, the decoder sequence can have a fourth labeling state: non-fluorescence, which is distinguishable from the three fluorescent labeling states above. The labeling state of a decoder sequence can be assigned to a digital value that can be conventionally used in an identification code for identifying a target sequence in the decoding process. For example, green, red, blue and no fluorescence labeling can be assigned a digital value of 1, 2, 3 and 0, respectively. The presence and the absence of a decoder sequence, which can be distinguished by a detection method, can be used as two labeling states in a decoding hybridization process. The labeling state of a DNA cluster is the same as that of the decoder sequence annealed to it in a decoding hybridization. During a decoding hybridization, the decoder sequence anneals to a complementary sequence in a DNA cluster and labels the DNA cluster with the same labeling state of the decoder sequence. For example, a decoder sequence with a labeling state of green fluorescence will label the DNA cluster of the complementary sequence with green fluorescence. If decoder sequences have only two labeling states, “presence” and “absence”, DNA clusters containing a DNA sequence complementary to a decoder sequence present in a decoding hybridization will be labeled as “presence”, while DNA clusters containing a DNA sequence complementary to none of the decoder sequences present in the decoding hybridization will be labeled as “absence”.

The term “identity of DNA clusters”, as used herein, refers to the identity of the DNA sequence that is contained in a physically distinguishable DNA cluster which is generated in a clonal amplification. Each DNA cluster comprises copies of identical sequences and occupies a physical location on a solid support. Each DNA cluster is defined by its physical location and the DNA sequence it contains. The DNA sequence within a DNA cluster is usually identified by a decoder sequence or detection sequences (e.g. a mutation detection sequence or a wild-type detection sequence) that can specifically recognize and bind to it. The identity of a DNA cluster can be identified as a target sequence including wild-type and mutant type alleles or a particular allele of the target sequence, depending on the decoder sequence used to decode it.

The term “decoding hybridizations”, as used herein, refers to sequential hybridization reactions of decoder sequence pools and the DNA clusters, which are used to decode the identity of DNA clusters immobilized to a solid support. Each round of decoding hybridization contains a different pool of decoder sequences which can be in different labeling states. The decoder sequences included in each pool is specified by the M-bit identification code of the decoder sequences.

The term “M-bit identification code”, as used herein, refers to a unique M-bit code that is used to represent and identify a target/decoder sequence in the decoding hybridization. The M-bit identification code contains information to specify the operation of the decoding hybridizations. M is calculated as ┌log_{N}T┐, which is the minimum number of decoding hybridization cycles required to decode T types of different target sequences when N is the total number of different labeling states for each decoder sequence. The i^{th} bit value (i=1, 2, . . . M) of the identification code specifies the labeling state of the target sequence specific decode sequence that is used in i^{th} round of decoding hybridization. For example, 6-bit identification codes are used to decode 50 sequences using decoder sequences having two labeling states: red and green fluorescence. The red and green fluorescence labeling is assigned a digital value of 1 and 2, respectively. A first decoder sequence having a 6-bit identification code of (121211) will be having a labeling state of Red, Green, Red, Green, Red and Red in the 1st, 2nd, 3rd, 4th, 5th and 6th round of decoding hybridization. A second decoder sequence having a 6-bit identification code of (221212) will be having a labeling state of Green, Green, Red, Green, Red and Green in the 1st, 2nd, 3rd, 4th, 5th and 6th round of decoding hybridization. After M rounds of decoding hybridizations, the labeling states order of a DNA cluster is compared with the M-bit identification codes for each decoder sequence. A DNA cluster is identified as having a target sequence when its labeling pattern matches to what is specified in the M-bit identification code of the target sequence. In the above example, a DNA cluster having the first decoder sequence specific target sequence will have labeling pattern of Red, Green, Red, Green, Red and Red in the 1st, 2nd, 3rd, 4th, 5th and 6th round of the decoding hybridization. A DNA cluster having the second decoder sequence specific target sequence will have labeling pattern of Green, Green, Red, Green, Red and Green in the 1st, 2nd, 3rd, 4th, 5th and 6th round of the decoding hybridization.

The term “decoder sequence pool”, as used herein, refers to a pool of selected decoder sequences labeled in different labeling states, which are used in decoding hybridization reactions to decode DNA clusters. For a decoding process that uses a M-bit identification code, there are a total of M pools of decoder sequences. The M-bit identification code of a decoder sequence defines whether the decoder sequence is included in a decoder sequence pool as well as the labeling state of the decoder sequence if included. For example, the first-round decoding hybridization uses a first pool of decoder sequences, each having a labeling state specified by the first bit value of its M-bit identification code. The second round decoding hybridization uses a second pool of decoder sequences, each having a labeling state specified by the second bit value of its M-bit identification code.

The term “mutation detection primer”, as used herein, refers to a DNA primer that comprises mutation specific sequence of a target sequence that is different from the wild-type sequence of the target sequence. For example, a mutation detection primer has one or more mutated nucleotides at the 3′ end which are not present in the wild-type sequence. The mutation detection primer preferably hybridizes to a mutant sequence and uses it as a template to make an extension strand. The mutation detection primer can't use wild-type sequence as a template to make an extension strand.

The term “wild-type detection primer”, as used herein, refers to a DNA primer that comprises wild-type specific sequence of a target sequence that is different from the mutant sequence of the target sequence. For example, a wild-type detection primer has one or more nucleotides at the 3′ end which are not present in the mutant sequence. The wild-type detection primer preferably hybridizes to a wild-type sequence and uses it as a template to make an extension strand. The wild-type detection primer can't use a mutant sequence as a template to make an extension strand.

The term “copy number variation”, as used herein, refers to the number of copies of a particular target region is different from a reference number of a reference region. The target region refers to a DNA or RNA sequence of interest that is suspected to have increased or decreased copy number from a normal reference number. The target region can be, for example, a single gene, a cDNA sequence, a genomic section, a subsection of a chromosome or a whole chromosome. The reference region is a DNA/RNA sequence or region that is known to have a normal copy number or a stable copy number, which can be used as a reference for comparison. The reference region can be, for example, a different region than the target region in the same sample. The reference region can also be the same as the target region in a different sample (e.g. a known normal sample). A copy number variation is detected when the copy number of a target region is significantly different from that of a reference region in the same sample. A copy number variation can also be detected by comparing a normalized copy number of a target region to a known reference copy number from different samples (e.g. normal samples). In some embodiment, the copy number variation can be construed to be differential expression of a target gene in a test sample vs. a normal sample.

The present invention provides a simple, robust and sensitive method for simultaneously detecting a large number of mutations of different target genes with high specificity. It exploits single-molecule clonal amplification techniques, a hybridization-based decoding technique and a primer extension-based detection method, allowing simultaneous measurement of hundreds and thousands of mutation DNAs in a sample. In the method, thousands and millions of DNA molecules in a sample are singly captured to a solid surface and are locally amplified to form immobilized DNA clusters of identical sequences. The DNA clustering having the target sequences are then identified by a decoding algorithm using sequential hybridization with a set of decoder sequence pools. Once decoded, the DNA clusters containing target sequences can be enumerated to determine the number of each target sequence in the sample. Alternatively, a pool of mutant specific primer can be simultaneously used to detect the presence of DNA mutations in the decoded DNA clusters.

The advantages of the invented method come from many different aspects. First, the quantitation of the amount of a target sequence in the sample is based on enumeration of DNA clusters having the target sequence, which is a digitalized method that does not depend on the absolute measurement value of labeling probes. Secondly, the single molecule clonal amplification can be performed on the DNA sample without pre-amplification, converting each original DNA molecule into a DNA cluster without the bias or distortion caused by an amplification process. Thirdly, the sensitivity for detecting target molecules is very high for this method. Theoretically, it can detect down to one single target molecule in a DNA sample. The detection of a DNA mutation is achieved by detection of labeled mutation specific strand generated by mutation specific primer extension. The specificity of the method lies at both the hybridization specificity of mutation specific primers and the selectivity of DNA polymerase that extends a matched 3′ end at a much higher efficiency than a mismatched 3′ end, which is much higher than detection methods that depend singly on probe hybridization specificity. Fourthly, the hybridization-based decoding technique is very efficient at identifying a large number of DNA clusters of target sequences, enabling simultaneous measurement of hundreds and thousands even millions of different types of target sequences without compromising the detection quality. In addition, since a decoder sequence plus detection sequences are hybridized to the same DNA cluster multiple times during the decoding and detection process, the invented method provides a mechanism for self-verification and confirmation, which further increase its accuracy and specificity. Fifthly, the invented method can use unlabeled sequence probes in combination with fluorescent nucleotides, circumventing the need of making hundreds and thousands of fluorescently labeled DNA probes. This can greatly reduce the material cost and the variation caused by difference in hybridization efficiency of different fluorescent probes. Additionally, the invented method is very versatile. It can be applied in a highly multiplexed format to detect DNA mutations or any sequences of interest, determine differential gene expression, and detect copy number variation. It can be applied to whole genome sequences, amplicon sequences, cDNAs, targeted sequences and cell free circulating DNA. Because of its high specificity, high sensitivity, high accuracy and highly multiplexed nature, it is especially suitable for detecting DNA mutations in circulating DNA samples and other clinical samples when the source materials are very limited.

The present invention provides a method for detecting copy number variation with high sensitivity and accuracy. The invention provides a method for efficiently and accurately counting thousands and millions of sequences from different target regions, enabling detection of copy number variation at the whole genome, the whole chromosome, sub-chromosomes or single gene level. The invented method is a more sensitive, specific and accurate yet less expensive alternative to the current microarray-based and high throughput sequencing-based technologies for detection of copy number variations.

In one embodiment, the present invention provides a method for simultaneously enumerating a plurality of target sequences in a DNA sample, comprising the steps of: a) performing a single-molecule clonal amplification on the DNA sample to obtain a large number of immobilized DNA clusters, each having an identical DNA sequence and being spatially separated from one another with a random distinguishable address; b) decoding the identity of the DNA clusters having target sequences by use of a hybridization decoding process with a set of decoder sequence pools; and c) enumerating DNA clusters having target sequences, thereby obtaining the number of each target sequence in the DNA sample. In some embodiment, it further comprises the steps of: a) denaturing and removing the decoder sequences from the DNA clusters; b) annealing a plurality of detection sequences to respective target sequences within the DNA clusters in a detection hybridization; c) labeling DNA clusters annealed to detection sequences; and d) enumerating labeled DNA clusters having target sequences. The detection sequence can be designed to be specific to an allelic form of a target sequence, for example, a mutation allele or a wild-type allele.

This method can be used to simultaneously detect a large number of mutation DNAs, or more generally any target DNA molecules with a unique sequence, in a DNA sample. The DNA sample can be prepared from cells, tissues, organs, soils, air, water, fossils and any other biological and environmental sources Particularly, a nucleic acid sample may be prepared from a patient's tissue, a body fluid, or cell samples such as urine, lymph fluid, spinal fluid, blood, and tumor cells/tissues, which can be used for clinical purposes. The starting material can be DNA or RNA.

The DNA and RNA can be extracted and purified from the source materials using standard purification methods known to an artisan skilled in the art of molecular biology (Current Protocol in Molecular Biology, Edited by Frederick M. Ausubel et al., John Weily and Sons, 2016; Sambrook et al., Molecular Cloning: A Laboratory Manual, Fourth Edition, Cold Spring Harbor Laboratories, New York, 2012). When the starting material is RNA, the RNA molecules can be converted to DNAs using reverse transcription reactions. The purified DNA sequences are then fragmented into 50-400 bp fragments, preferably 70-250 bp fragments, or more preferably 100-200 bp fragments using techniques well known in the art, for example, enzymatic digestion, sonication, mechanical shearing, electrochemical cleavage, and nebulization. The DNA fragments of appropriate sizes are selected and connected to sequence tags on both ends. The sequence tags are designed sequences that are non-complementary to all the sequences in the nucleic acid sample. The methods to add sequence tags to the ends of DNA fragments are well known in the art, which usually includes DNA repair, end polishing and sequence tag ligation. In some embodiments, the sequence tags can be added to the DNA fragments by PCR amplification. The PCR-free tagging method is preferable as it produces a tagged DNA population without sequence coverage bias associated with the PCR steps. The sequence tags on each end of the DNA fragment can have the same or different sequences, but all the DNA fragments share the same sequence tags. The doubled tagged DNA sample is then ready to be used in the clonal amplification reaction to generate DNA clusters of identical sequences.

The single molecule clonal amplification technique is used to generate spatially distinguishable clusters of a large number of DNA copies of a single DNA molecule from the DNA sample. The clonal amplification technique allows capturing and amplifying of a single DNA molecule and fixing the amplified molecules to a localized address. Each DNA cluster and a DNA molecule in the sample has a 1-to-1 corresponding relationship. Thus, detecting features of DNA clusters allows detection limit down to single molecule level. Several clonal amplification methods are suitable for use in the invented method, including, for example, polony technology (J. Shendure et al. Science 309, 1728-1732 (2005); and H. V. Chetverina, & A. B. Chetverin Nucleic Acids Res. 21, 2349-2353 (1993)); beads, emulsion, and amplification magnetics (BEAM) (D. Dressman, et al.. Proc. Natl. Acad. Sci. USA 100, 8817-8822 (2003)); emulsion polymerase chain reaction (emPCR) (M. Margulies, et al. Nature 437, 376-380 (2005); M. J. Embleton, et al. U.S. Pat. No. 5,830,663; and A. Griffiths & D. Tawfik, U.S. Pat. No. 6,489,103); a cloning strategy developed for massively parallel signature sequencing (MPSS) (S. Brenner, et al. Proc. Natl. Acad. Sci. USA 97, 1665-1670 (2000)); and the bridge PCR amplification scheme (C. Adessi, et al. PCT patent application WO2000018957; and T. C. Boles, et al. U.S. Pat. No. 5,932,711).

In some embodiment, double tagged DNA sequences are clonally amplified on channels of a glass side/flow cell using a Bridge PCR. Briefly, the surface of the flow cell is printed with two types of oligonucleotide primers that are complementary to 3′ and 5′ sequence tags on the DNA molecules, respectively. A single DNA molecule anneals to one oligonucleotide primer and allows extension of the oligonucleotide primer to make a complementary copy of the DNA molecule by DNA polymerase mediated polymerization. The duplex DNA is denatured and the unattached DNA strand is removed from the flow cell surface. The attached DNA strand has the complementary sequence of the original DNA molecule with two sequence tags. Under appropriate annealing conditions, the unattached sequence tag bends over and anneals to the neighboring oligonucleotide, and use the neighboring oligonucleotide as primer to make another complementary DNA strand, which has the same sequence of the original DNA molecule. The duplex DNA is denatured and allows two attached single-stranded DNA molecule to serve as a template for next cycle of PCR amplification. This in situ PCR process can be repeated many times until a cluster of thousands and millions of DNA sequence copies are generated. The concentration of the DNA sample and the cycle number of PCR can be optimized so that each DNA cluster comprises a population of identical sequences and complementary sequences and is spatially separate from neighboring clusters. The DNA clusters are first generated with two complementary sequences and will form a duplex under non-denaturing conditions. To make single-stranded DNA clusters, one of the two complementary sequences is removed. This is achieved by introducing a cleavable site on each of the oligonucleotide primers. The cleavable sites on two oligonucleotide primer is distinct from each other so that each strand can be cleaved selectively, leaving another strand intact. The cleavable sites can be made to be, for example, photocleavable, chemically cleavable, or enzymatically cleavable.

In some embodiment, double tagged DNA sequences are clonally amplified on microbeads or other microparticles using an emulsion PCR as described in Margulies, M. et al. Nature 437, 376-380 (2005). Briefly, the DNA molecules are ligated to a sequence tag with a biotin incorporated on one strand. DNA molecules are bound to streptavidin beads under conditions that favor one DNA per bead. The beads are captured in the droplets of a PCR-reaction-mixture-in-oil emulsion and PCR amplification occurs within each droplet, resulting in beads each carrying ten million copies of a unique DNA template. The emulsion is broken, the DNA strands are denatured, and beads carrying single-stranded DNA clones are deposited into wells of a fiber-optic slide.

In another embodiment, double tagged DNA sequences are clonally amplified on microbeads attached with two types of the oligonucleotide primers using an emulsion PCR as described in YM Xu, et al. (Biotechniques. 48(5):409-412. (2010)). Briefly, the two types of the oligonucleotide primers are attached to the surface of microbeads. The double-tagged DNA molecules are annealed to the oligonucleotide primers of the microbeads under conditions favoring one molecule per bead. The beads are captured in the droplets of PCR-reaction-mixture-in-oil emulsion and PCR amplification occurs within each droplet, resulting in beads carrying both complementary strands of the original DNA sequence. One strand of the two complementary sequences are removed using the methods described above.

In another embodiment, the single-molecule clonal amplification is conducted in thousands and millions of premade wells on a microchip. The wells are pretreated to have the 3′ and 5′ sequence tags attached to the surface. The tagged DNA sequences are distributed to the wells under the condition that no more than one single molecule is deposited into one well. Perform a Bridge-PCR amplification in each well to generate a DNA cluster of identical sequences in the well.

After generation of thousands and millions of clonal DNA clusters on a solid surface, the next step is to identify DNA clusters having target sequences using a hybridization-based decoding process. Once the identity of DNA clusters having target sequences is determined, that is, physical addresses for DNA clusters containing a target sequence are correctly located on the solid support, a pool of allele specific detection sequences/primers are added to the decoded DNA clusters to simultaneously detect the DNA clusters having the particular allelic form of each target sequence. It should be noted that not the identity of every DNA cluster on the solid support is decoded by the decoding process, only those DNA clusters having target sequences are identified by the decoder sequences recognizing the target sequences.

In some embodiment, the detection primer is specific to a mutant allele of the target sequence. This method can be used to enumerate mutant alleles of a plurality of target sequences in the DNA sample. The mutant specific detection primer comprises a different sequence from the decoder sequence which recognizes both the mutant and wild-type allele of the target sequence. The mutant specific detection primer contains at least one mutated nucleotide at its 3′ end, which renders it to preferably anneal to a mutant allele over a wild-type allele. With the aid from DNA polymerase's selectivity to preferably add a nucleotide to a perfectly matched over a mismatched 3′ end, the mutant primer directed DNA extension offers much higher detection specificity than direct hybridization. After the decoding process, a pool of mutation specific primers are added to the DNA clusters and annealed to complementary sequences within the DNA clusters. A DNA polymerase and a dNTP mix with at least one type of labeled nucleotides are added to the reaction system, extending annealed mutation specific primer to generate labeled extension strands. The DNA clusters having a labeled extension strand is identified as the DNA cluster having a mutant sequence. Since the identity of DNA clusters associated with the target sequences is known during the decoding process, the DNA clusters of mutant sequences for each target sequence can be determined. The mutant allele frequency for a target sequence can be calculated by dividing the number of DNA clusters having a mutant allele by the number of DNA clusters having the target sequence. In another embodiment, the mutation specific primer extension is detected by chemical or physical signals generated during the DNA polymerization process, including, but not limited to, detection of pyrophosphates, hydrogen ions or temperature changes.

After DNA clusters with the mutant alleles are identified, the extension strands can be denatured and removed from the DNA clusters, and wild-type specific detection primers can be added to detect wild-type alleles. In some embodiment, the method further comprising the steps of: a) denaturing and removing the extension strands from the DNA clusters, and annealing a plurality of wild-type detection primers of the target sequences to respective complementary sequences within the DNA clusters; b) labeling the DNA clusters annealed to a detection primer using detection primer mediated DNA polymerization; c) enumerating labeled DNA clusters with the decoded identity, thereby simultaneously counting the number of DNA molecules of the wild-type allele for each target sequence; and d) calculating a mutant allele frequency for each target sequence by dividing the number of the mutant allele with the total number of the mutant and the wild-type allele.

In some embodiment, the detection primer is specific to a wild-type allele of the target sequence and the method is used to enumerate wild-type alleles of different target sequences in a DNA sample. This method can be used to detect, for example, differential expression of target genes and copy number variation of a chromosome or subsections of a chromosome. In some embodiment, the decoder sequence for a target sequence is the same as the detection primer for the same target sequence. In another embodiment, the decoder sequence for a target sequence is different from the detection primer for the same target sequence. For example, the decoder sequence can be in close proximity with the detection sequence or overlap or be a part of the detection sequence. Using detection primer and detection by extension method to detect decoded target sequences can further verify the accuracy of the decoding process and increase the detection specificity. A decoding sequence pool does not necessarily contain all the decoder sequences in a decoding hybridization whereas all the detection sequences are included in the detection process. The decoding process can use different probe sequences and detection methods than those of the detection process. Combining both the decoding and the detection process can greatly decrease the error rate and increase the accuracy and specificity of the final result.

In some embodiment, the method is used for detection of copy number variation of target sequences. To detect copy number variation of a plurality of target sequences, the target sequences are grouped into a first part of the target sequences which are sequences to be tested for the presence of a copy number variation, and a second part of the target sequences which are reference sequences that are known to have no copy number variation. Decoder sequences specific for the first and the second part of the target sequences are used for the decoding process. The presence of copy number variation for a target sequence is detected when the number of the target sequence is significantly different from those of reference sequences.

In some embodiment, the decoding process comprises the steps of: a) providing a decoder sequence specific for each target sequence, wherein each decoder sequence has N different labeling states, wherein N is at least 2; b) designing a M-bit identification code to uniquely represent each decoder sequence, wherein M rounds of decoding hybridizations are to be performed to decode T types of different target sequences, and the value of i^{th} bit (i=1, 2, . . . M) of the M-bit identification code of a decoder sequence defines the labeling state of the decoder sequence used in the decoder sequence pool for the round decoding hybridization, wherein M is ┌log_{N}T ┐, and T is the number of different types of target sequences; c) making a set of M pools of decoder sequences according to the M-bit identification codes; d) performing M rounds of decoding hybridizations with the decoder sequences pool set and DNA clusters in an order defined by the M-bit identification codes; and e) recording the labeling state of each DNA cluster in each round of decoding hybridization to decode the identity of DNA clusters based on the M-bit identification code for each decoder sequence. In some embodiment, an additional (M+1)^{th} round hybridization with a selected decoder sequence pool can be used to verify the decoding accuracy.

In order to simultaneously count a large number of different target sequences, a decoding algorithm needs to be applied to identify which DNA cluster contains which target sequence. The decoding algorithm makes use of hybridizations of different combinations of target sequence specific decoder sequences that are labeled in different states to figure out the identity of all the DNA clusters containing target sequences. For a total number, T, of all the different types of target sequences and a total number, N, of all different labeling states that each decoder sequence has, the minimum number of hybridization reactions required to decode T different types of target sequences is M=┌log_{N}T┐. The key of this decoding algorithm is to use a unique M-bit identification code to represent each target sequence and to direct M rounds of decoding hybridizations. The M-bit identification code is designed such that the i^{th} bit of the identification code represents the i^{th} decoding hybridization reaction and the i^{th} bit value of the identification code defines the labeling state of the respective decoder sequence in the decoding hybridization. In some embodiment, the labeling state of a decoder sequence is represented by the type of the label (e.g. fluorophore type) linked to the decoder sequence, wherein the total number (N) of different labeling states can be selected from 2, 3, 4, 5, 6, 7 or more. For example, the decoder sequences can be labeled with a red, a green or a blue fluorescent moiety and the number N of different labeling states is three. A decoder sequence pool can include every decoder sequences. But the labeling states of the decoder sequences is different for different decoder sequence pools. In some embodiment, the labeling state of a decoder sequence is represented by the type of the fluorophore linked to the decoder sequence, and with an additional labeling state, non-fluorescence. The decoder sequence of non-fluorescence is the one not included in a particular decoder sequence pool.

To illustrate how the decoding algorithm works, 15 decoder sequences with two different fluorescent labelings are used. The minimum number of hybridization reactions needed to decode 15 different target sequences is 3=┌log₃15┐. The 3 different labeling states for each decode sequence are digitally designated as 1 (red), 2 (green), and 0 (non-fluorescence). No fluorescence means that the particular decoder sequence is not included in the particular pool. An exemplary set of M-bit identification codes designed for decoding 15 target sequences are shown in Table 1.

TABLE 1
Design of 3-bit identification code for decoding
15 sequences

| | 3-bit identification code | | |
|---|---|---|---|
| Target Sequence No. | 1st bit | 2nd bit | 3rd bit |
| | 1 | 0 | 0 |
| | 2 | 0 | 0 |
| | 0 | 1 | 0 |
| 1 | 1 | 1 | 0 |
| 2 | 2 | 1 | 0 |
| | 0 | 2 | 0 |
| 3 | 1 | 2 | 0 |
| 4 | 2 | 2 | 0 |
| | 0 | 0 | 1 |
| 5 | 1 | 0 | 1 |
| 6 | 2 | 0 | 1 |
| 7 | 0 | 1 | 1 |
| 8 | 1 | 1 | 1 |
| 9 | 2 | 1 | 1 |
| 10 | 0 | 2 | 1 |
| 11 | 1 | 2 | 1 |
| 12 | 2 | 2 | 1 |
| | 0 | 0 | 2 |
| 13 | 1 | 0 | 2 |
| 14 | 2 | 0 | 2 |
| 15 | 0 | 1 | 2 |
| | 1 | 1 | 2 |
| | 2 | 1 | 2 |
| | 0 | 2 | 2 |
| | 1 | 2 | 2 |
| | 2 | 2 | 2 |

Three rounds of decoding hybridizations are needed for decoding 15 target sequences. An example of selecting 3-bit identification codes for 15 target sequences is shown in the Table 1. The 3-bit identification code of “000” should not be used as it cannot distinguish a target sequence from the non-target sequences that have no positive signals. To increase the specificity and lowering the error detection rate, each identification code is chosen to have at least two fluorescent labeling states. As shown in the above example, the first, the second and the third pool of decoder sequences used in the decoding hybridization are set in the following lists: (1,2,1,2,1,2,0,1,2,0,1,2,1,2,0), (1,1,2,2,0,0,1,1,1,2,2,2,0,0,1), and (0,0,0,0,1,1,1,1,1,1,1,1,2,2,2), respectively, wherein the position No. in the list represents the corresponding decoder sequence No., and the bit value of the identification code defines the labeling state of respective decoder sequence (e.g. 1 for red, 2 for green, and 0 for no fluorescence). After three rounds of decoding hybridization using the first, the second and the third decoder sequence pool, all the DNA clusters containing a target sequence can be identified by comparing labeling pattern of DNA clusters to the 3-bit identification codes. For example, the No. 1 target sequence has a 3-bit identification code as (1,1,0) and all the DNA clusters having the No. 1 target sequence should be labeled as red, red, no fluorescence in the three sequential decoding hybridizations. The No.2 target sequence has a 3-bit identification code as (2,1,0) and all the DNA clusters having the No. 2 target sequence should be labeled as green, red, no fluorescence in the three sequential decoding hybridizations.

In some embodiment, the decoder sequence comprises of two oligonucleotides that form a donor-receptor pair of a fluorescence resonance energy transfer. The two oligonucleotides are designed to be complementary to adjacent sections of the same target sequence, wherein the two oligonucleotides are end-labeled with fluorophores to form a 3′ donor-5′ acceptor or a 5′ donor-3′ acceptor pair. Only when both oligonucleotides bind to the target sequence and the donor and the acceptor fluorophore are in close proximity, the energy emitted from the donor fluorophore can excite the acceptor fluorophore. Using a FRET donor-acceptor pair as a decoder sequence can increase the hybridization specificity. The methods for designing a FRET donor-acceptor oligonucleotide pair are well known in the art (V. V. Didenko, Biotechniques. 2001; 31(5): 1106-1121). The general requirements include that the emission spectrum of the donor fluorophore needs to overlap with the absorbance spectrum of the acceptor fluorophore, and the donor and acceptor fluorophore should be brought to close proximity (e.g. 1-10 nm apart) so that the energy transfer can occur efficiently.

In some embodiment, a decoder sequence has two different labeling states, represented by the presence and the absence of the decoder sequence, respectively. This embodiment does not use the label on a decoder sequence to distinguish different labeling states of a decoder sequence, but use the presence or absence of a decoder sequence to distinguish them. To implement this approach, a decoder sequence pool contains only a part of all the decoder sequences. The presence of a decoder sequence is designated as 1 and the absence of a decoder sequence is designated as 0 in the M-bit identification code, and each decoder sequence is represented by a M-bit binary identification code. Each decoder sequence pool comprises a selected combination of decoder sequences that is determined by the M-bit identification codes for the decoder sequences.

In some embodiment, the presence of a decoder sequence is detected by a label directly linked to the decoder sequence. The label linked to the decoder sequence is a biotin, a fluorophore, or a chemiluminescent moiety. In some embodiment, all the decoder sequences are labeled with the same fluorophore. In some embodiment, the decoder sequences are labeled with different fluorophores. fluorophore. In some embodiment, the decoder sequences can be labeled with different fluorophores. For example, half of the decoder sequences are labeled a red fluorophore, and the other half of decoder sequences are labeled with a green fluorophore. Labeling the decoder sequences with different fluorophores help differentiate DNA clusters and decrease the decoding error rate. All the labeled DNA clusters indicate the presence of a decoder sequence and have a labeling state value of 1 irrespective of the labeling color. In some embodiment, the decoder sequence comprises of two oligonucleotides complementary to adjacent sections of its target sequence, wherein the two oligonucleotides are respectively labeled with a donor and an acceptor fluorophore of a fluorescence resonance energy transfer pair.

Using labeled probes for the decoding process can become very expensive when very high throughput assays are needed. For example, if more than a million of decoder sequences are needed for an assay, the cost of making labeled decoder sequences can become prohibitively expensive. In some embodiment, decoder sequences are unlabeled, and the presence of a decoder sequence is detected by decoder sequence specific DNA extension. Using the presence and the absence of a decoder sequence as two labeling states and detecting the presence of a decoder sequence using decoder sequence specific DNA polymerization circumvent the need of making thousands and millions of labeled sequence probes. As an added advantage, detection method based on sequence specific extension has higher specificity than hybridization-based detection method because the former relies on both the specificity of DNA hybridization and the selectivity of DNA polymerase for extending a matched over a mismatched 3′ end. In some embodiment, an unlabeled decoder sequence pool, a DNA polymerase, and a dNTP mix with a fluorescent nucleotide are added during a decoding hybridization, and the presence of a decoder sequence in a DNA cluster is detected by the decoder sequence specific extension that makes a labeled extension strand. In some embodiment, one, two, three or four types of nucleotides in the dNTP mix are substituted by respective fluorescent nucleotides.

In some embodiment, nucleotides with two different fluorophores are alternately used to label decoder sequence specific extension strands in sequential decoding hybridizations so as to detect erroneous decoding instances. For example, during the decoding hybridizations, a green and a red fluorescent nucleotide are used to alternately label the decoder specific extension strands. The second decoding hybridization should have a different labeling color than that of the first hybridization. If the color of the first hybridization is found in the second hybridization, a detection error is found. Using different fluorescent nucleotides to label the extension strands can thus help detect decoding errors and non-specific labeling.

In some embodiment, an unlabeled decoder sequence pool, a DNA polymerase, and a dNTP mix of four natural nucleotides are added during the decoding hybridization. The presence of a decoder sequence in a DNA cluster is detected by recording a chemical or physical change generated by the decoder sequence specific DNA extension. The chemical or physical change generated by the decoder sequence specific DNA extension includes, but not limited to, pyrophosphates, H⁺ ions, and temperature change.

In some embodiment, the present invention provides a method for simultaneously enumerating a plurality of different target sequences in a DNA sample, comprising the steps of: a) performing a single-molecule clonal amplification on the DNA sample to obtain a large number of immobilized DNA clusters of identical DNA sequences, wherein each DNA cluster is spatially separated from one another and has a random distinguishable address; b) providing a plurality of decoder sequences, each specific for a target sequence, wherein each decoder sequence has two labeling states, the presence of the decoder sequence and the absence of the decoder sequence, which are assigned digital values of 1 and 0, respectively; c) designing a M-bit binary identification code to uniquely represent a decoder sequence, wherein M rounds of decoding hybridizations are to be performed to decode T types of decoder sequences, and the value of i^{th} bit (i=1, 2, . . . M) of the M-bit identification code defines the labeling state of the respective decoder sequence used in the decoder sequence pool for the i^{th} round decoding hybridization, wherein M is ┌log₂T ┐, and T is the number of different types of decoder sequences; d) making a set of M decoder sequence pools according to the M-bit binary identification codes; e) performing M rounds of sequential decoding hybridizations with the decoder sequences pool set and the DNA clusters in an order defined by the M-bit identification codes, wherein labeling states of DNA clusters in each round of decoding hybridization are determined by decoder sequence mediated DNA polymerization to make extension strands; and f) recording the labeling state of each DNA cluster in each round of decoding hybridization to decode the identity of DNA clusters and count the number of each target sequence in the DNA sample.

In some embodiment, target sequences are mutant sequences of a plurality of target genes and the decoder sequences comprise mutant specific sequences. This method can be used to directly count mutant sequences of different target sequences in a DNA sample.

In some embodiment, the target sequences are separated into a first part comprising mutant sequences of target genes and a second part comprising corresponding wild-type sequences of the target genes. Accordingly, the decoder sequences are separated into the first part of decoder sequences comprising mutant specific sequences and the second part of decoder sequences comprising wild-type specific sequences. This method can be used to detect both the mutant and wild-type alleles of the target genes and calculate mutant allele frequency for each target gene. It uses decoder sequences specific for mutant or wild-type allele to decode and count the numbers of mutant and wild-type alleles. A additional (M+1)^{th} of hybridization with a selected decoder sequence pool can be used to verify the correctness of the decoding result and further increase the detection specificity.

In some embodiment, the presence of a decoder sequence is determined by decoder sequence mediated DNA polymerization that makes a labeled extension strand. The detection of labeled strands in a DNA cluster indicates that the DNA cluster comprises a sequence complementary to a decoder sequence. To make a labeled extension strand, a labeled dNTP is added with DNA polymerase and other dNTPs during decoder sequence mediated DNA polymerization. The labeled extension strand can comprise, for example, a fluorescent, a chemiluminescent or a biotin label. In some embodiment, one, two, three or four types of fluorescent nucleotides are added during decoder sequence mediated DNA polymerization.

In some embodiment, the presence of a decoder sequence is determined by detecting a physical or chemical change generated by decoder sequence mediated DNA polymerization. The physical or chemical change is selected from pyrophosphate, hydrogen ion and temperature change generated during decoder sequence mediated DNA polymerization.

In some embodiment, the method is used for detection of copy number variations (CNV). The target sequences are separated into a first part of target sequences that are to be tested for presence of copy number variations and a second part of target sequences which are reference sequences known to have no copy number variation. Accordingly, the decoder sequences are separated into the first part of decoder sequences comprising first target sequence specific sequences and the second part of decoder sequences comprising reference sequence specific sequences. The number of each target sequence can be determined after M round of decoding hybridizations. The presence of a copy number variation for a target sequence is detected when the number of the target sequence in the DNA sample is significantly different from those of reference sequences.

The present invention provides a method for simultaneously measuring hundreds, thousands, even millions of different DNA sequences, which is especially suitable for detecting copy number variations at gene level, chromosome level or whole genome level. With only two labeling states, a hundred, a thousand and a million different DNA species can be decoded using 7, 10 and 20 hybridization reactions, respectively. The decoder sequences can be easily designed to target to genes of interest, subsections of a chromosome, a target chromosome and genome-wide sequences. The DNA sequences from the reference region and the target region of the same DNA sample can be measured in the same assay, which avoids the requirement of cross-sample comparisons and greatly increases the accuracy of the detection assay. The method directly counts the number of DNA molecules randomly captured and clonally amplified from the DNA sample, thus providing a truthful representation of the distribution of DNA molecules in the original sample with minimum bias and distortion. With the high sensitivity, specificity and accuracy, the invented method can satisfy the requirement of detecting fetal DNA copy number variations from maternal cell-free circulating DNA samples.

In some embodiment, the present invention provides a method for detecting copy number variation of a plurality of different target regions of a DNA sample, comprising the steps of: a) providing a plurality of first decoder sequences, each complementary to a different target sequence within one of the target regions, and providing a plurality of second decoder sequences, each complementary to a different target sequence within one of reference regions; b) performing a single-molecule clonal amplification on the DNA sample to obtain a large number of immobilized DNA clusters of identical DNA sequences, wherein each DNA cluster is spatially separated from one another and has a random distinguishable address; c) combining the first and the second decoder sequences to decode DNA clusters having sequences complementary to the first or second decoder sequences using the decoding method described above; d) counting the number of each target sequence of target regions and the number of each target sequence of reference regions; and e) comparing the numbers of target sequences of target regions and the numbers of target sequences of reference regions to determine if a target region has a copy number variation. The presence of copy number variation of a target region is detected when the numbers of target sequences of the target region are significantly different from those of the reference regions. Alternatively, a normalized count of a target region can be obtained by dividing the average number of target sequences of the target region by those of reference regions, which can be used to be compared with a standard value to determine the presence of a copy number variation.

In some embodiment, the invented method is used to detect copy number variations in genomic regions of interest (e.g. disease-related genes). The decoder sequences are designed to be complementary to target sequences of target regions of interest and to target sequences of reference regions that are known to have no copy number variations. Count numbers of target sequences from target regions and those from the reference regions. Detect a copy number variation of a target region when the average sequence count from the target region is significantly different from that of the reference region.

In some embodiment, the invented method is used to determine if a target chromosome has a copy number variation (e.g. triploid). The decoder sequences can be designed to distribute evenly along a chromosome or to be targeted to the stable regions of a chromosome. The number of decoder sequences can be at least at least 20, 30, 50, 100, 200, 500, 1000, 10000, or 100000. In some embodiment, the average number of all the target sequences of the target chromosome and the average number of all the target sequences of the reference chromosome are used to detect the occurrence of a copy number variation. If the average number of the target sequences of the target chromosome is significantly different from that of the reference chromosome, it is determined that the target chromosome has a copy number variation.

In some embodiment, target sequences of a chromosome are grouped into a sequence bin of certain length and the average number of target sequences in each sequence bin for the target and the reference chromosome are used for determination of the presence of a copy number variation in the target chromosome. The length of a sequence bin can be at least 10 kb, 100 kb, 1 Mb, or 10 Mb. If the average number of the target sequences in each sequence bin of the target chromosome is significantly different from that of the reference chromosome, it is determined that the target chromosome has a copy number variation.

In some embodiment, the invented method is applied to detect copy number variations at the whole genome level. The decoder sequences can be chosen to be evenly distributed across the whole genome. The number of decoder sequences needed depends on the detection resolution required. For example, 100 thousand, 1 million or 10 million decoder sequences can be selected to give a coverage of one decoder sequence in every 30 kb, 3 kb and 300 bp, respectively. In some embodiment, the detection of the genome wide copy number variations can be performed in two stages. In the first stage, decoder sequences are designed to identify broad potential regions of copy number variation at the whole genome level. Once the possible regions of copy number variation are identified, decoder sequences specifically targeting to those regions can be designed to further verify and delineate the size and range of the CNV regions. For example, 100 thousand decoder sequences that are evenly distributed across the whole genome are first used to identify possible CNV regions as small as 30 kb. A possible CNV region is defined as a region having at least one decoder sequence count significantly different from the average count of all the decoder sequences. Secondly, decoder sequences are designed to specifically target regions around the possible CNV regions (e.g. 100 decoder sequences per region). The decoder sequences for possible CNV regions along with known reference decoder sequences are used to further refine the detection of CNV regions. Using the two step methods, a lot less decoder sequences can be used to detect genome-wide CNVs with a great resolution.

### EXAMPLES

The invention is further illustrated in more details with reference to the accompanying examples. It is noted that, the following embodiments are only intended for purposes of illustration and are not intended to limit the scope of the invention.

### Experiment 1. Simultaneous Detection of Fifty Somatic Mutations in a Cell-Free Circulating DNA Sample

This example demonstrates how to use the invented method to detect multiple somatic mutations in cell-free circulating DNA (cfNA) samples.

Preparation of Decoder Sequences and Mutation Detection Primers

Before starting testing the sample, design 50 mutation detection primers with 3′ end having at least one mutated nucleotide and 50 decoder sequences that overlap with the mutation detection primer at 5′ sequences without the 3′ mutated nucleotides. The decoder sequence is either labeled with a red or a green fluorescence label. The length of mutation detection primers is 20 to 25 nt.

DNA Extraction and Tagging

A cfDNA sample is extracted from a patient's blood using a commercially available extraction kit such as MagMAX Cell-Free DNA Isolation Kit (Thermo Fisher Scientific, Waltham, Mass.) and QIAamp circulating nucleic acid kit (Qiagen, Valencia, Calif.).

A double-tagged DNA preparation is made from the extracted cfDNA using an illumina-compatible NGS sample preparation kit such as NEBNext® Ultra™ II DNA Library Prep Kit for Illumina® (NEB, Ipswich, Mass.) and Truseq DNA PCR-free library preparation kit (Illumina, San Diego, Calif.). The DNA sequences made from these preparation kit have two different sequence tags at the 3′ and 5′ ends, which can be used as common anchors to attach the DNA sequences to the oligonucleotides immobilized on a flow cell (a specially made glass slide).

Clonal Amplification for DNA Cluster Generation

The double-tagged DNA molecules are used as templates for generation of millions of DNA clusters. The cluster generation is performed in the Illumina® flow cell on a cBot instrument (Illumina, San Diego, Calif.), which involves immobilization and 3′ extension, bridge amplification and linearization. The outcome product is millions of clonal clusters each with about 1000 single-stranded DNA molecules covalently attached on the surface of the flow cell.

Decoding the DNA Clusters Having Target Sequences

A hybridization-based decoding algorithm is used to identify the DNA clusters having one of fifty target sequences. The red fluorescent (value:1) and the green fluorescent (value:0) of a decoder sequence are the two labeling states that are used to decode 50 target sequences. The total number of decoding hybridizations used is 6 (┌log₂50┐). A red fluorescent decoder sequence is included in the decoder sequence pool of a particular round of decoding hybridization if the bit value of the decoder sequence for the particular round is 1, and a green fluorescent decoder sequence is included if the bit value is 0. The 6-bit identification codes are selected such that red fluorescent decoder sequence is included at least two times in the six hybridization reactions. The 6-bit identification codes for each target sequence are selected as shown in Table 2. For example, the 6-bit identification codes for target sequence No. 1, No. 2 and No. 3 are 110000, 101000, and 011000, respectively. The decoder sequence pools for six rounds of decoding hybridization are shown in the columns for 1st bit, 2nd bit, 3rd bit, 4th bit, 5th bit and 6th bit.

The six rounds of decoding hybridize reaction is performed as follows: adding the first pool of decoder sequences to hybridize with the DNA clusters; measuring the fluorescence of each DNA cluster and assigning a digital value to each DNA cluster based on fluorescence readout (Red: 1, Green: 0); denaturing and removing the bound decoder sequences; adding the second pool of decoder sequences to perform the second round decoding hybridization; repeating the decoding hybridizations until all the six rounds of hybridization reactions are completed. The identity of each DNA cluster having a target sequence can be determined by comparing the fluorescence readout pattern with the 6-bit identification code. For example, the fluorescence readout pattern for target sequence No. 1 should be “Red/Red/Green/Green/Green/Green” as defined by the 1st 6-bit identification code (110000). The fluorescence readout pattern for target sequence No. 2 should be “Red/Green/Red/Green/Green/Green” as defined by the 2nd 6-bit identification code (101000).

TABLE 2
6-bit identification code table for decoding 50 sequences

| target | 6-bit identification code | | | | | |
|---|---|---|---|---|---|---|
| sequence No. | 1st bit | 2nd bit | 3rd bit | 4th bit | 5th bit | 6th bit |
| 1 | 1 | 1 | 0 | 0 | 0 | 0 |
| 2 | 1 | 0 | 1 | 0 | 0 | 0 |
| 3 | 0 | 1 | 1 | 0 | 0 | 0 |
| 4 | 1 | 1 | 1 | 0 | 0 | 0 |
| 5 | 1 | 0 | 0 | 1 | 0 | 0 |
| 6 | 0 | 1 | 0 | 1 | 0 | 0 |
| 7 | 1 | 1 | 0 | 1 | 0 | 0 |
| 8 | 0 | 0 | 1 | 1 | 0 | 0 |
| 9 | 1 | 0 | 1 | 1 | 0 | 0 |
| 10 | 0 | 1 | 1 | 1 | 0 | 0 |
| 11 | 1 | 1 | 1 | 1 | 0 | 0 |
| 12 | 1 | 0 | 0 | 0 | 1 | 0 |
| 13 | 0 | 1 | 0 | 0 | 1 | 0 |
| 14 | 1 | 1 | 0 | 0 | 1 | 0 |
| 15 | 0 | 0 | 1 | 0 | 1 | 0 |
| 16 | 1 | 0 | 1 | 0 | 1 | 0 |
| 17 | 0 | 1 | 1 | 0 | 1 | 0 |
| 18 | 1 | 1 | 1 | 0 | 1 | 0 |
| 19 | 0 | 0 | 0 | 1 | 1 | 0 |
| 20 | 1 | 0 | 0 | 1 | 1 | 0 |
| 21 | 0 | 1 | 0 | 1 | 1 | 0 |
| 22 | 1 | 1 | 0 | 1 | 1 | 0 |
| 23 | 0 | 0 | 1 | 1 | 1 | 0 |
| 24 | 1 | 0 | 1 | 1 | 1 | 0 |
| 25 | 0 | 1 | 1 | 1 | 1 | 0 |
| 26 | 1 | 1 | 1 | 1 | 1 | 0 |
| 27 | 1 | 0 | 0 | 0 | 0 | 1 |
| 28 | 0 | 1 | 0 | 0 | 0 | 1 |
| 29 | 1 | 1 | 0 | 0 | 0 | 1 |
| 30 | 0 | 0 | 1 | 0 | 0 | 1 |
| 31 | 1 | 0 | 1 | 0 | 0 | 1 |
| 32 | 0 | 1 | 1 | 0 | 0 | 1 |
| 33 | 1 | 1 | 1 | 0 | 0 | 1 |
| 34 | 0 | 0 | 0 | 1 | 0 | 1 |
| 35 | 1 | 0 | 0 | 1 | 0 | 1 |
| 36 | 0 | 1 | 0 | 1 | 0 | 1 |
| 37 | 1 | 1 | 0 | 1 | 0 | 1 |
| 38 | 0 | 0 | 1 | 1 | 0 | 1 |
| 39 | 1 | 0 | 1 | 1 | 0 | 1 |
| 40 | 0 | 1 | 1 | 1 | 0 | 1 |
| 41 | 1 | 1 | 1 | 1 | 0 | 1 |
| 42 | 0 | 0 | 0 | 0 | 1 | 1 |
| 43 | 1 | 0 | 0 | 0 | 1 | 1 |
| 44 | 0 | 1 | 0 | 0 | 1 | 1 |
| 45 | 1 | 1 | 0 | 0 | 1 | 1 |
| 46 | 0 | 0 | 1 | 0 | 1 | 1 |
| 47 | 1 | 0 | 1 | 0 | 1 | 1 |
| 48 | 0 | 1 | 1 | 0 | 1 | 1 |
| 49 | 1 | 1 | 1 | 0 | 1 | 1 |
| 50 | 0 | 0 | 0 | 1 | 1 | 1 |

Detection of DNA Mutations

The pool of 50 mutation specific primers, Taq DNA polymerase, a 4-dNTP mixture with dTTP being substituted by fluorescent dUTP are added to the flow cell after the decoding process. Using mutant sequences as the templates, the Taq DNA polymerase catalyzes the extension of the 3′ end of the mutant specific primers and incorporates the fluorescent nucleotides into a mutant specific extension strand. Take a fluorescence image to record the number, fluorescence intensity, and the location of labeled DNA clusters to determine the number of the mutant sequences for each target sequences in the DNA sample. The mutant allele frequency of a target sequence can be calculated by dividing the number of the mutant allele by the number of the target sequence.

### Experiment 2. Simultaneous Detection of 200 Genomic Mutation Sequences Using a Detection by Extension Decoding Method

This method demonstrates how to simultaneously measure 200 genomic mutations using a detection by extension decoding method.

Design and prepare 200 decoder sequences specific for mutant sequences and 200 decoder sequences specific for the corresponding wild-type sequences of target sequences.

Prepare DNA samples use a genomic DNA preparation kit and perform clonal amplification to make DNA clusters as described in Example 1.

Perform the decoding process to identify DNA clusters containing a mutant target sequence or a wild-type target sequence using the 400 decoder sequences above. The decoding process uses the presence (value:1) and absence (value: 0) of decoder sequences as two labeling states. The minimum number of decoding hybridizations needed is 9 ([log₂400]). To examine the accuracy of the decoding result, add a 10th hybridization to test if the decoded assignment of each DNA cluster is correct. Make a 9-bit identification table for 400 decoding sequences as shown in Example 1. To increase decoding specificity, each identification code should have at least 3 positive labeling states, that is, every decoder sequence should be used at least 3 times in the 10 hybridizations. The decoder sequences are not labeled in this example, and the presence of a decoder sequence is detected by decoder sequence specific DNA extension. In each decoding hybridization, a pool of selected decoder sequences, a DNA polymerase, a dNTP mixture with dTTP substituted by fluorescent dUTP are added together. Labeled DNA extension strands are only made in DNA clusters having sequences complementary to a decoder sequence. Record the labeling states of each DNA cluster in 9 decoding hybridizations and compare the labeling pattern to the 9-bit identification codes to decode each DNA cluster. When a DNA cluster has a labeling pattern matching to a particular identification code of a decoder sequence, the DNA cluster is identified as containing a sequence complementary to the decoder sequence. In the 10th hybridization, the labeling states of the decoded DNA clusters are compared to the expected labeling value. If the labeling state of a DNA cluster matches the expected value, it is confirmed to be a correct decoding assignment. Otherwise, the decoding assignment is not correct and the decoded DNA cluster will not be included in the final result.

After the verified decoding process, the numbers of 200 mutant sequences and 200 wild-type sequences in the DNA sample can be determined. The mutant allele frequency of a target sequence can be calculated by dividing the number of the mutant allele by the total number of the mutant and wild-type allele of the target sequence.

### Experiment 3. Simultaneous Detection of 100 Copy Number Variations Using FRET Labeled Decoder Sequences

This method demonstrates how to simultaneously detect 100 copy number variations in a cell-free circulating DNA sample.

Design and prepare 30 decoder sequences for each target region that has a potential copy number variation to obtain a total of 3000 decoder sequences for 100 target regions. Design and prepare 30 unlabeled decoder sequence for each of 10 reference region that is known to have no copy number variation to obtain 300 decoder sequences for reference regions. The decoder sequence comprises two oligonucleotides which are complementary to adjacent regions of the same target sequence. The 5′ of the upstream oligonucleotide is labeled with a green donor fluorophore and the 3′ of the downstream oligonucleotide is labeled with a red acceptor fluorophore. Only when both oligonucleotides bind to the target sequence, can the energy transfer between the donor and accepter fluorophore occur. This FRET-based decoder sequence can greatly increase the specificity of detection.

Prepare DNA samples and perform clonal amplification to make DNA clusters as described in Example 2.

Perform the decoding process as the follows. To decode 3300 different types of decoder sequences using the presence (1) and absence (0) of decoder sequences as two labeling states, the minimum number of decoding hybridizations needed is 12=┌log₂3300┐. To examine the accuracy of the decoding process, add a 13th hybridization to test if the decoded assignment of each DNA cluster is correct. Make a 12-bit identification table for 3300 decoder sequences as shown in Example 1. To increase decoding specificity, each identification code should have at least four positive labeling states, that is, every decoder sequence should be used at least four times in the 12 hybridizations. The presence of a decoder sequence in a DNA cluster is detected by the light emitted from the FRET donor-acceptor pair of the annealed decoder sequence.

After the decoding process, the numbers of decoder sequence specific sequences for 100 targeted regions and 10 reference regions can be determined. Compare the average counts for each target region to that of the reference regions to determine if a target region has a copy number variation.

### Experiment 4. Detection of Genome-Wide Copy Number Variation in a Genomic DNA Sample

This method demonstrates how to detect genome-wide copy number variations in a genomic DNA sample.

Design and prepare 10 million unlabeled decoder sequences to evenly cover the whole genome on an average of one decoder sequence in every 300 bp.

Prepare DNA samples use genomic DNA preparation kit and perform clonal amplification to make DNA clusters as described in Example 1.

Perform the decoding process as the follows. To decode 10 million different types of decoder sequences using the presence (1) and absence (0) of decoder sequences as two labeling states, the minimum number of decoding hybridizations needed is 24. To examine the accuracy of the decoding process, add a 25th hybridization to test if the decoded assignment of each DNA cluster is correct. Make a 24-bit identification table for 10 million decoder sequences as shown in Example 1. To increase decoding specificity, each identification code should have at least ten positive labeling states, that is, every decoder sequence should be used at least ten times in the 24 hybridizations. The decoder sequences are not labeled in this example, and the presence of a decoder sequence is detected by decoder sequence specific DNA extension and incorporation labeled nucleotides into the extension strand as shown in Example 2.

After the decoding process, the numbers of 10 million decoder sequence specific sequences can be determined. Calculate the average count for each decoder sequence specific sequence and look for genomic regions that have significantly lower or higher count than the average count of the whole genome. The genomic regions with significantly lower or higher count are determined to the ones with a copy number variation.

While the present invention has been described in some detail for purposes of clarity and understanding, one skilled in the art will appreciate that various changes in form and detail can be made without departing from the true scope of the invention. All figures, tables, appendices, patents, patent applications and publications, referred to above, are hereby incorporated by reference.

What is claimed is:

1. A method for simultaneously enumerating a plurality of target sequences in a DNA sample, comprising the steps of:
a) performing a single-molecule clonal amplification on the DNA sample to obtain a large number of immobilized DNA clusters, each having an identical DNA sequence and being spatially separated from one another with a random distinguishable address;
b) decoding the identity of the DNA clusters having target sequences by use of a hybridization decoding process with a set of decoder sequence pools; and
c) enumerating DNA clusters having target sequences, thereby obtaining the number of each target sequence in the DNA sample.

2. The method of claim 1, wherein the hybridization decoding process comprises the steps of:
a) providing a decoder sequence specific for each target sequence, wherein each decoder sequence has N different labeling states, wherein N is at least 2;
b) designing a M-bit identification code to uniquely represent each decoder sequence, wherein M rounds of decoding hybridizations are to be performed to decode T types of different target sequences, and the value of i^{th} bit (i=1, 2, . . . M) of the M-bit identification code of a decoder sequence defines the labeling state of the decoder sequence used in the decoder sequence pool for the i^{th} round decoding hybridization, wherein T is the total number of different types of target sequences and M is no less than ┌log_{N}T┐;
c) making a set of M pools of decoder sequences according to the M-bit identification codes;
d) performing M rounds of sequential decoding hybridizations with the decoder sequence pool set and the DNA clusters in an order defined by the M-bit identification codes; and
e) recording the labeling state of each DNA cluster in each round of decoding hybridization to decode the identity of DNA clusters based on the M-bit identification code for each decoder sequence.

3. The method of claim 2, wherein different alleles of a target sequence are recognized by one target sequence specific decoder sequence.

4. The method of claim 2, wherein different alleles of a target sequence are recognized by different allele specific decoder sequences.

5. The method of claim 2, wherein the labeling state of a decoder sequence is represented by the type of the detectable label linked to the decoder sequence.

6. The method of claim 2, wherein the labeling state of a decoder sequence is represented by the type of the detectable label linked to the decoder sequence and with an additional labeling state represented by no presence of the decoder sequence.

7. The method of claim 2, wherein the decoder sequence comprises two oligonucleotides complementary to adjacent sections of its target sequence, wherein the two oligonucleotides are respectively end labeled with a donor and an acceptor fluorophore that form a FRET pair.

8. The method of claim 2, wherein the decoder sequence has two labeling states, represented by the presence and the absence of the decoder sequence, respectively.

9. The method of claim 8, wherein each decoder sequence pool comprises a selected combination of decoder sequences, wherein the presence of a decoder sequence is designated as 1 and the absence of a decoder sequence is designated as 0 in the M-bit identification code, and each decoder sequence is represented by a M-bit binary identification code.

10. The method of claim 9, wherein decoder sequences are unlabeled, and the presence of a decoder sequence is detected by decoder sequence mediated DNA polymerization.

11. The method of claim 2, further comprising the steps of:
f) denaturing and removing the decoder sequences from the DNA clusters;
g) annealing a plurality of detection sequences to respective target sequences within the DNA clusters in a detection hybridization;
h) labeling DNA clusters annealed to detection sequences; and
i) enumerating labeled DNA clusters having target sequences.

12. The method of claim 11, wherein the decoder sequence and the detection sequence of a target sequence is the same.

13. The method of claim 11, wherein the decoder sequence and the detection sequence of a target sequence is different.

14. The method of 13, wherein the decoder sequence is target sequence specific and the detection sequence is allele specific.

15. The method of claim 2, wherein the method is used for detection of copy number variation of the target sequences, wherein the target sequences are divided into a first and second part, wherein the first part contains sequences to be tested for the presence of copy number variation, and the second part contains reference sequences that are known to have no copy number variation, and wherein the presence of a copy number variation for a target sequence is detected when the number of the target sequence is significantly different from those of reference sequences.

16. The method of claim 2, wherein the method is used for detecting copy number variation of a plurality of different target regions of a DNA sample, wherein the decoder sequences are divided into a plurality of first decoder sequences, each complementary to a different target sequence within one of the target regions, and providing a plurality of second decoder sequences, each complementary to a different target sequence within one of reference regions that are known to have no copy number variation, wherein the first and the second decoder sequences are combined to use for decoding the DNA Clusters, and wherein the numbers of target sequences of a target region and the numbers of target sequences of reference regions are compared to determine if the target region has a copy number variation.

17. The method of claim 16, wherein the target region is a genomic region of interest, a cDNA sequence, a chromosome or a whole genome.

18. The method of claim 16, wherein the average number of all the target sequences of a target region and the average number of all the target sequences of a reference region is used to determine if the target region has a copy number variation.

19. The method of claim 16, wherein target sequences of a target region are grouped into a sequence bin of certain length, and the average number of target sequences in each sequence bin of the target region and the average number of target sequences in each sequence bin of the reference region are used for determination of the presence of copy number variation in the target region.
